(19) United States
(12) Patent Application Publication
(10) Pub. No.: US 2023/0149271 A1
(43) Pub. Date: May 18, 2023

(54) TOOTHPASTE COMPOSITION

(71) Applicant: LG HOUSEHOLD & HEALTH CARE LTD., Seoul (KR)

(72) Inventors: Ji Young KIM, Seoul (KR); Ji Eun YANG, Seoul (KR); Kyo Tae MOON, Seoul (KR); Jong Hyun LIM, Seoul (KR)

(73) Assignee: LG HOUSEHOLD & HEALTH CARE LTD., Seoul (KR)

(21) Appl. No.: 16/978,952

(22) PCT Filed: Mar. 6, 2020
(86) PCT No.: PCT/KR2020/003184

§ 371 Date: Sep. 8, 2020

(51) Int. Cl. A61K 8/22; A61Q 11/00; A61K 8/21; A61K 8/27; A61K 8/20; A61K 8/24; A61K 8/19; A45D 40/24

(52) U.S. Cl. CPC A61K 8/22; A61Q 11/00; A61K 8/21; A61K 8/27; A61K 8/20; A61K 8/24; A61K 8/19; A45D 40/24; A61K 2800/92; A61K 2800/88; A61K 2800/87; A61K 2800/48; A61K 2800/28; A45D 2200/055

(57) ABSTRACT

A toothpaste composition for tooth whitening comprising first contents and second contents, wherein one of the first contents and second contents comprises a tooth whitening agent, and it is constructed such that the first contents and second contents are filled in a container having a diaphragm or separate containers and simultaneously used when brushing teeth is provided. The container is a dip tube container.

### TECHNICAL FIELD

The invention relates to a whitening toothpaste composition for use in a dip tube pumping container.

### BACKGROUND ART

With the increase in interests in personal hygiene including oral cavity health, consumers who intend to obtain effects more than tooth brushing through daily used toothpaste or gargle rather than using a separate product for oral cavity health are increasing. Meanwhile, although active ingredients assisting in oral cavity health have excellent effects when used alone, when used in combination with common ingredients of toothpaste, the effects may decrease or long term storage stability may be lowered.

Recently, with the increase in interests in tooth whitening as well as oral cavity health, toothpaste compositions having tooth whitening effects are being released. Representative active ingredients for tooth whitening include peroxide, which has been used in the dentist for more than 100 years, and of which whitening effect has been proved. Peroxide acts as a reducing agent for oxidizing tooth coloration material, and modifies or decomposes to remove coloration material, thereby achieving tooth whitening, but has a limitation in storage due to high reactivity. And, a polishing agent is commonly added to a toothpaste composition for washing, such a polishing agent has low compatibility with peroxide, thus rendering commercialization difficult. In order to secure marketability of a toothpaste composition, peroxide was included in a low concentration or a stabilizer was added in an excessive amount, which decreased the whitening effect of the toothpaste composition.

Previously, in case whitening toothpaste comprises peroxide in the concentration of 3% or more, it was classified into a high concentration whitening toothpaste, and examples thereof included a product using stabilized peroxide gel referred to as a booster or accelerator together with paste type toothpaste. This product was inconvenient because two tubes should be respectively squeezed on toothbrush.

Another example included a product for brushing teeth with common paste type toothpaste, and then, brushing teeth with gel containing peroxide without washing with water, but the directions was inconvenient due to the need to brush teeth in two steps.

Meanwhile, toothpaste comprising stannous fluoride has antigingivitis, anticavity and desensitization effects. However, stannous fluoride is easily hydrolyzed and oxidized in water to become stannous ion(Sn⁴⁺) and lose its activity. And, stannous fluoride toothpaste may bond to sulfide in the oral cavity to cause coloration. In order to stabilize stannous fluoride and obtain desired effects, it is preferable to apply in the form of low water toothpaste having water content below a certain level or anhydrous toothpaste, but the low water or anhydrous toothpaste has low preference of the user due to bitterness and fever of polyol.

On the other hand, in the oral cavity health, mechanisms for preventing or alleviating sensitive teeth include nerve desensitization by potassium nitrate, protein precipitation by glutaraldehyde, silver nitrate, zinc chloride, strontium chloride, and the like, and plugging dental tubules using sodium fluoride, stannous fluoride, strontium chloride, silver diamine fluoride, potassium oxalate, calcium phosphate, calcium carbonate, bioactive glasses, monobasic potassium phosphate, dibasic potassium phosphate, and the like. Previously, it was expected that synergistic effect may be obtained if different ingredients inducing different mechanisms are used together, but practically, if these ingredients are added together to a toothpaste composition, before plugging dental tubules or causing protein precipitation, precipitate is formed in the composition, and thus, proper effects could not be obtained.

As such, previously, toothpaste compositions that contain whitening ingredients assisting in oral cavity health, antigingivitis ingredients, desensitizer ingredients, and the like, but do not have an influence on the usual tooth brushing habit of consumers, and thus, are not inconvenient or cumbersome have not been provided.

### DISCLOSURE

### Technical Problem

In order to solve the problems of the prior art, it is an object of the invention to provide a toothpaste composition comprising active ingredients for oral cavity health, which is suitable for separately storing the contents in a two-chamber container according to compatibility, and then, discharging and using them.

It is another object of the invention to provide a toothpaste composition comprising active ingredients for oral cavity health, which has properties convenient to store in a two-chamber container of dip tube type, and then, discharge and use.

### Technical Solution

The invention relates to a toothpaste composition for tooth whitening comprising first contents and second contents, wherein one of the first contents and second contents comprises a tooth whitening agent, it is constructed that the first contents and second contents are filled in a container having a diaphragm or separate containers and simultaneously used when brushing teeth, and the container is a dip tube container.

In the toothpaste composition according to the invention, the first contents may comprise peroxide, and the second contents may comprise a polishing agent.

Specifically, the peroxide may be one or more selected from the group consisting of urea peroxide, sodium peroxide, calcium peroxide and hydrogen peroxide.

Specifically, at least one of the first contents and the second contents may comprise a thickener.

Specifically, at least one of the first contents and the second contents comprises the thickener in the amount of 20 wt % or less, based on 100 wt % of each contents.

Specifically, the thickener may be one or more selected from the group consisting of precipitated silica, fumed silica, alumina, talc, calcium carbonate, poloxamer, xanthan gum, carbomer, carboxymethyl cellulose, polyacrylic acid, polyvinyl pyrrolidone, polyethylene, hydroxypropyl methylcellulose, and hydroxypropyl cellulose.

Specifically, the second contents may comprise the polishing agent in the amount of 5 to 20 wt %, based on 100 wt % of the second contents.

In the toothpaste composition according to the invention, the first contents may comprise one or more selected from the group consisting of stannous fluoride, zinc chloride, potassium nitrate, calcium chloride and strontium chloride, and the second contents may form a complex with the above ingredients or inactivate the above ingredients.

Specifically, the first contents may comprise stannous fluoride, and the second contents comprise at least one of pyrophosphoric acid, polyphosphoric acid, and pharmaceutically acceptable salts thereof and water, and comprise water in the amount of 20 to 60 wt % based on 100 wt % of the second contents.

Specifically, the first contents may further comprise water, and comprise water in the amount of 2 to 10 wt %, based on 100 wt % of the first contents.

Specifically, the second contents may comprise at least one of pyrophosphoric acid, polyphosphoric acid, and pharmaceutically acceptable salts thereof in the content of 2 to 12 wt %, based on 100 wt % of the second contents.

Specifically, the first contents may comprise one or more selected from the group consisting of zinc chloride, potassium nitrate, calcium chloride and strontium chloride, and the second contents may comprise one or more selected from the group consisting of disodium succinate, dicalcium oxalate, monopotassium phosphate, and dipotassium phosphate.

Specifically, the viscosities of the first contents and the second contents may be 5,000 to 40,000 cps.

Specifically, the ratio of the viscosities of the first contents and the second contents may be 1.2:1 to 1:1.2.

Specifically, the container may comprise a container body for providing a storage space of the contents, a tube for inhaling the contents, a pump, and a nozzle for discharging the contents.

Specifically, the container may further comprises a diaphragm for dividing the internal space, and the first contents and the second contents may be respectively stored in a first storage space and a second storage space divided by the diaphragm.

Specifically, the container may comprise a first container in which the first contents are stored, and a second container in which the second contents are stored.

Specifically, the first container and the second container may be combined so as to be separatable from each other.

Specifically, the second container may be disposed in the first container.

Specifically, the tube may comprise a first tube for inhaling the first contents; and a second tube for inhaling the second contents, and the nozzle may simultaneously discharge the first contents and the second contents when brushing teeth, through one or more outlets.

### Advantageous Effects

The toothpaste composition according to one embodiment of the invention comprises first contents comprising peroxide as a tooth whitening agent and ingredients compatible with peroxide, and second contents comprising a polishing agent, and is stored in a container having a diaphragm or separate dip tube containers and simultaneously used when brushing teeth, thus enabling stable storage of peroxide included in the toothpaste composition.

The toothpaste composition according to one embodiment of the invention comprises stannous fluoride as the first contents, thus providing antigingivitis effect, and simultaneously, comprises at least one of pyrophosphoric acid, polyphosphoric acid and pharmaceutically acceptable salts thereof as the second contents, thus preventing coloration by stannous fluoride and providing tooth whitening effect.

The toothpaste composition according to one embodiment of the invention comprises one or more selected from the group consisting of zinc chloride, potassium nitrate, calcium chloride and strontium chloride as the first contents, thus providing desensitization effect, and simultaneously, comprises one or more chelating agents selected from the group consisting of disodium succinate, dicalcium oxalate, monopotassium phosphate, and dipotassium phosphate as the second contents, thus forming a complex with the ingredients of the first contents to provide tooth whitening effect. And, since the contents are not mixed before use, precipitation is not generated beforehand.

The tooth paste composition according to the embodiments of the invention provides tooth whitening effect by at least one of the first contents and second contents, the viscosities of the first contents and second contents are suitable for apply in a dip tube type container, and the viscosity and rate of each contents are maintained within a certain range even if time elapses according to storage. Thus, hardening at the outlet of the dip tube container is prevented over the period of storage of the toothpaste composition, and each contents can be simultaneously discharged.

### DESCRIPTION OF DRAWINGS

FIG. 1a is front view of a two-chamber container and FIG. 1b is a side view of the two-chamber container according to one embodiment of the invention.

FIG. 2a shows the two-chamber container with the nozzle removed, and FIG. 2b is a cross-sectional view of the inside of the container body, in the two-chamber container of FIGS. 1a and 1b.

FIG. 3a is a front view of a two-chamber container and FIG. 3b is a side view of the two-chamber container according to one embodiment of the invention.

FIG. 4a shows the two-chamber container with the nozzle removed, and FIG. 4b is a perspective view when the container is decoupled, in the front view of the two-chamber container of FIG. 3.

FIGS. 5a and 5b are cross-sectional views of the two-chamber container according to one embodiment of the invention.

FIGS. 6a and 6b are cross-sectional views of the two-chamber container according to one embodiment of the invention.

FIG. 7a is a front view of the two-chamber container according to one embodiment of the invention, and FIG. 7b is a front view showing that the cap is removed.

FIG. 8a is a front cross-section view and FIG. 8b is a top plan view of the two-chamber container of FIG. 7.

### BEST MODE

The purpose, specific advantages and novel features of the invention will be more apparent from the detailed description and preferable examples below. However, there examples are presented only as the illustrations of the invention, and the scope of the invention is not limited thereby. And, in the explanation of the invention, in case it is judged that specific explanations of related known technology may unnecessarily cloud the gist of the invention, detailed explanations thereof will be omitted.

As used herein, tooth whitening means removal of material that attaches or penetrates on the surface of teeth and inside of enamel to cause coloration, or modification of the chemical structure of corresponding material to remove coloration.

First, referring to attached drawings, a toothpaste container for storing the toothpaste composition according to the invention will be explained.

The toothpaste container for storing a toothpaste composition is a two-chamber container. Hereinafter, the two-chamber container means a container having a diaphragm inside of one container or separate containers, providing storage spaces for two contents.

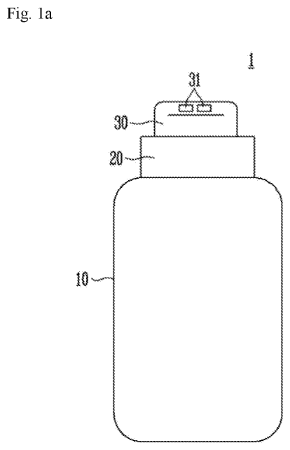

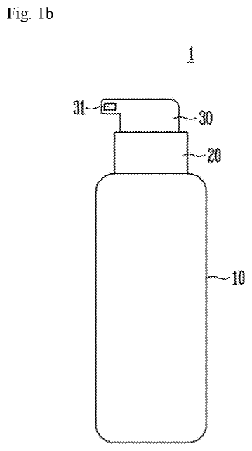

Referring to FIGS. 1a to 6b, the two-chamber container (1) according to one embodiment of the invention will be explained.

Referring to FIGS. 1a and 1b, the two-chamber container (1) may be a dip tube container comprising a container body (10), a tube (50), a pump (40) and a nozzle (30).

The container body (10) provides a storage space for storing contents. The shape of the container body (10) is not limited, and it may comprise a polymer such as polyethylene terephthalate, polyethylene, polypropylene, polyethylene naphthalate, poly(butylenes 2,6-naphthalate), polychloritrifluoroethylene, polytetrafluoroethylene, and the like, or metal, but not limited thereto.

The tube (50) may be disposed so as to be extended to the bottom of the container or spaced apart at a predetermined distance, and connect the contents. In the tube (50), the cross-section of one end inhaling the contents may be oval, but the shape is not limited thereto.

A fixing member (20) provides a space for accommodating a cap (21) described below inside. And, the fixing member (20) is coupled with a nozzle (30) to provide a space capable of up and down movement as the user pushes the nozzle (30).

The nozzle (30) may be used as a push part for pumping, and the user may push the nozzle to operate the pump and discharge the contents to the outside of the container through one or more outlets (31) provided in the nozzle. However, as shown in FIGS. 1a and 1b, the shape and length of the nozzle (30) are not limited. For example, the nozzle (30) may be provided with a length providing a sufficient distance such that a toothbrush may not be bumped against the container body (10) when the user touches a toothbrush head with the outlet (31) and pushes the nozzle. And, the outlet (31) may be disposed in the downward direction rather than at the side of the nozzle (30), and thus, when the user pushes the nozzle (30) while touching a toothbrush head with the outlet (31) or placing it in the adjacent position, the toothpaste may be stably discharged.

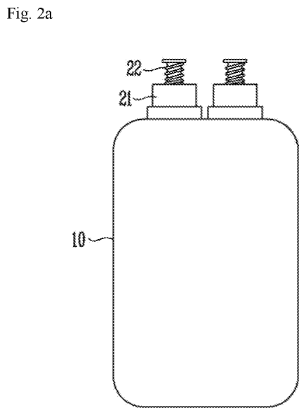

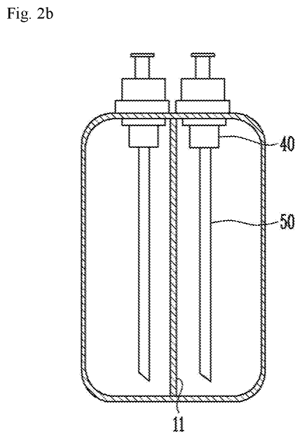

Referring to FIGS. 2a and 2b, the two-chamber container (1) may have a diaphragm (11) inside of one container body (10), thus providing two contents storage spaces. The diaphragm (11) may prevent different two contents from being mixed in the container, before the contents are discharged. The diaphragm (11) may divide the internal space of the container into a first storage space in which the first contents are stored and a second storage space in which the second contents are stored, and the shape or position in the container of the diaphragm (11) are not limited. Thus, the shapes and volumes of two contents storage spaces divided by the diaphragm (11) may or may not be identical to each other.

In each of the first storage space and second storage space, a tube (5) and a pump (40) may be disposed. For example, in the first storage space, a first tube, and in the second storage space, a second tube may be disposed. The user may push the nozzle (30) to pump both pumps (40), thus simultaneously discharging two contents.

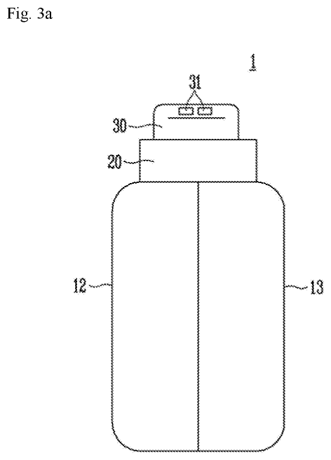

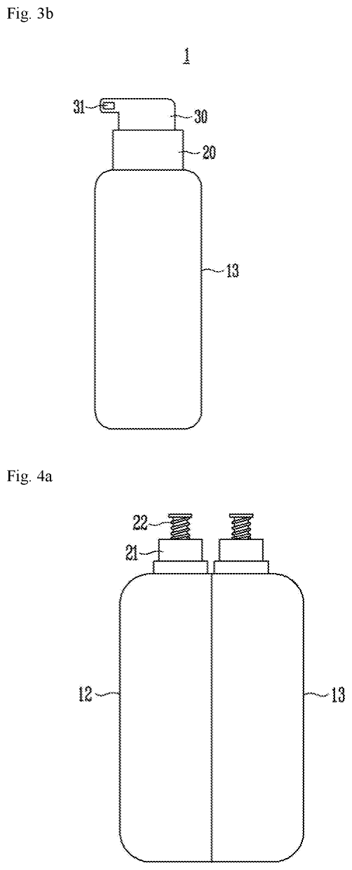

Referring to FIGS. 3a and 3b, the two-chamber container (1) may be separate dip tube containers providing two storage spaces. The two-chamber container (1) may comprise a first container (12) and second container (13) combined so as to be separatable from each other.

For example, the first container (12) and second container (13) may be dip tube containers respectively storing the first contents and second contents. The two containers may have identical or different shapes.

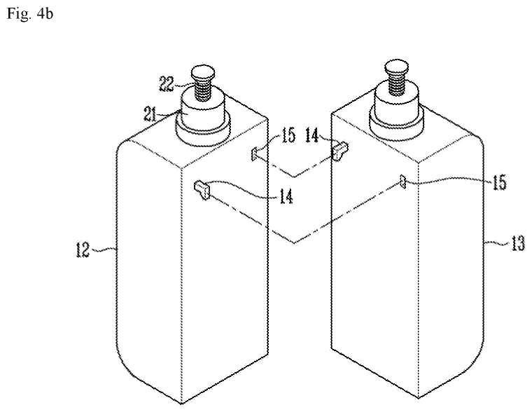

Referring to FIGS. 4a and 4b, at least one of the first container (12) and second container (13) may comprise a coupling member for coupling the other container on the outside. The coupling member may comprise a protrusion part (14) provided outside of the container, and a coupling groove (15) provided so as to be fitted according to the shape of the protrusion part (14). Referring to FIG. 4b, each of the first container (12) and second container (13) has a protrusion part (14) and a coupling groove (15), the protrusion part (14) of the first container (12) is fitted to the coupling groove (15) of the second container (13), and the protrusion part (14) of the second container (13) is fitted to the coupling groove (15) of the first container (12).

For another example, the first container (12) and second container (13) constituting the two-chamber container (1) may be coupled so as to be separated by a fixing member (20). Although not shown, the fixing member (20) comprises a space capable of accommodating a cap (21) provided in each container, and it may separatably fix the cap (21).

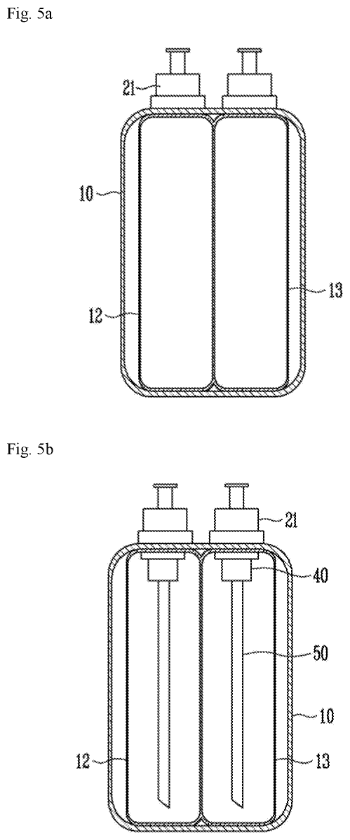

Referring to FIGS. 5a and 5b, the two-chamber container (1) may have a relatively small sized first container (12) and a second container (13) disposed in the container body (10). The first container (12) and second container (13) may be dip tube containers, and the two containers may have identical or different shapes.

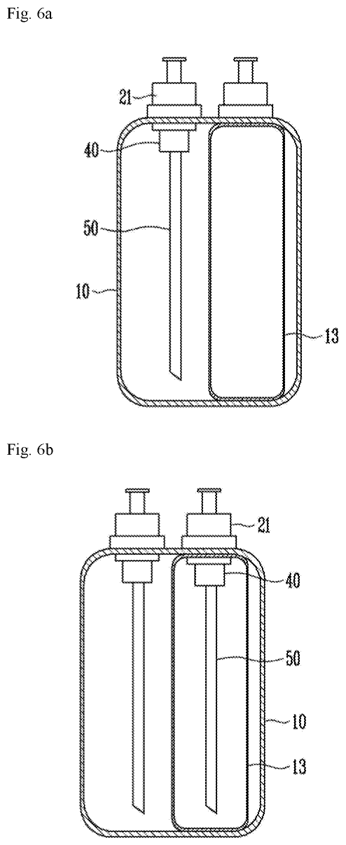

And, referring to FIGS. 6a and 6b, the two-chamber container (1) may have a relatively small sized second container (13) disposed in the container body (10). On end of the second container (13) may be coupled to the container body (10).

In the container body (10), a space except the second container (13) is provided as a first storage space, and the space in the second space (13) may be provided as a second storage space. In this case, the second container (13) functions as a diaphragm dividing the contents storage space of the container body (10) into two spaces. The first storage space and second storage space may have identical or different volume. In the container body (10), a pump (40) and a tube (50) may disposed in the first storage space may be disposed, and in the second container (13), a pump (40) and a tube (50) disposed in the second storage space may be disposed.

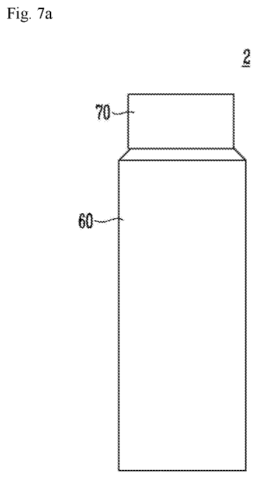

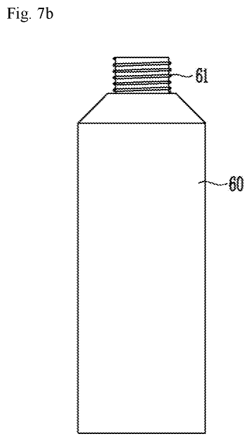

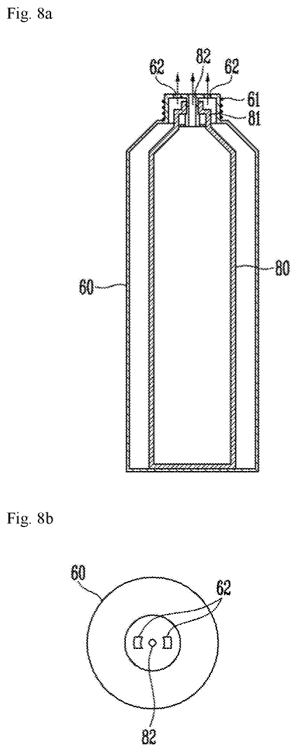

Referring to FIGS. 7a to 8b, the two-chamber container (2) according to one embodiment of the invention will be explained.

Referring to FIGS. 7a and 7b, the two-chamber container (2) may be a squeeze type tube container comprising a first container body (60), a first discharge part (61) and a cap (70).

Referring to FIGS. 8a and 8b, the two-chamber container (2) may be a tube-in-tube container in which a relatively small sized second container body (80) is disposed in a first container body (60). The first container body (60) and second container body (80) may have identical or different shapes.

The second container body (80) comprises a second discharge part (81) for discharging the contents, and one end of the second discharge part (81) may be coupled to the first discharge part (61) of the first container body (60). As shown in FIG. 8(a), the first discharge part (61) may be fitted to the second outlet (82) that is formed in the second discharge part (81) and discharges the contents.

In the first container body (60), a part except the second container body (80) may be provided as a first storage space, and the space in the second container body (80) may be provided as a second storage space. The first contents stored in the first storage space may be discharged through one or more first outlets (62) provided at one end of the first discharge part (61). The second contents stored in the second storage space may be discharged through the second discharge part (81) and discharged through the second outlet (82) provided in the first discharge part (61). As the user holds the first container body (60) in hand and squeezes it, the first container body (60) and second container body (80) are pushed and the stored first and second contents may be simultaneously discharged.

However, the shape and length of the first discharge part (61) and second discharge part (81) are not limited as shown in FIGS. 7a to 8b. For example, each discharge part may be provided in a shape having narrower cross section toward the end. Thereby, the user may easily discharge toothpaste even on a narrow toothbrush head. And, the shape and number of the first outlet (62) and second outlet (82) are not limited.

As used herein, a toothpaste composition having excellent dischargeability from a dip tube container and suitable for pumping means that it is discharged in an amount appropriate for tooth brushing, when a common user applies a predetermined force to a commercially available dip tube container.

As used herein, stable or storage stable peroxide means that the remaining rate of peroxide included in a toothpaste composition is 80% or more, when the toothpaste composition is put in a dip tube container and stored at 50° C. for 4 weeks. Peroxide having excellent stability means that the remaining rate of peroxide is 90% or more under the above described conditions.

As used herein, shape retention of toothpaste means the shape and retention of toothpaste when pumping a toothpaste composition, and toothpaste without shape retention means that toothpaste does not remain on the surface of toothbrush and flows between toothbrush head or below toothbrush within 10 seconds after discharging the toothpaste composition.

As used herein, a toothpaste composition constructed so as to be simultaneously used when brushing teeth means that the first contents and second contents of the toothpaste composition are simultaneously discharged. A simultaneously discharged toothpaste composition means that as the push part of a dip tube container is pushed to pump, the first contents and second contents are simultaneously pumped and the amounts thereof appear to be the same when observing with the unaided eye. For example, the colors of the first contents and second contents may be different, and the contents of two colors are simultaneously discharged as the toothpaste composition is used.

As used herein, loosening of toothpaste means that a toothpaste composition is loosened without maintaining a mass shape in the oral cavity when pumping the toothpaste composition on a toothbrush and brushing teeth. Excellent loosening of toothpaste means that when brushing teeth, the toothpaste composition is loosened within 30 seconds in the oral cavity and toothpaste in the form of mass does not flow in the oral cavity.

The toothpaste composition according to one embodiment of the invention is a toothpaste composition for tooth whitening comprising first contents and second contents, wherein one of the first contents and second contents comprises a tooth whitening agent, it is constructed such that the first contents and second contents are filled in a container having a diaphragm or separate containers and simultaneously used when brushing teeth, and the container is a dip tube container.

The first contents and second contents may be an oral composition for use in a toothpaste composition, and any one of the first contents and second contents may comprise a tooth whitening agent.

In the toothpaste composition according to one embodiment of the invention, the tooth whitening agent may be peroxide. In this case, the toothpaste composition according to the embodiment of the invention may comprise first contents comprising peroxide, and second contents comprising a polishing agent. The peroxide may one or more selected from the group consisting of urea peroxide, sodium peroxide, calcium peroxide and hydrogen peroxide. In case the first contents comprise peroxide, the peroxide may be included in the amount of 1 to 30 wt %, based on 100 wt % of the first contents. Considering the total amount of the toothpaste composition, the peroxide may be included in the amount of 0.5 to 15 wt %, based on 100 wt % of the toothpaste composition. Preferably, the peroxide may be included in the amount of 0.5 to 6 wt %, more preferably in the amount of 0.5 to 5 wt %, based on 100 wt % of the toothpaste composition. If the content of peroxide is less than 0.5 wt %, desired whitening effect may not be obtained, and if it is greater than 15 wt %, side effects such as tooth hypersensitivity or irritation in the oral cavity may be induced.

In case peroxide is used as a tooth whitening agent, one or more polyhydric alcohol such as glycerin, polyethylene glycol such as PEG300, polypropylene glycol, and the like may be used as a solvent for dissolving peroxide. The polyhydric alcohol may be included in the amount of 20 to 60 wt %, based on 100 wt % of the first contents. If the polyhydric alcohol is included in the amount less than 20 wt %, relatively excessive amount of water may be included in the toothpaste composition, and thus, water in the toothpaste composition may be evaporated in a dip tube container connecting with the air.

In case peroxide is used as a tooth whitening agent, a stabilizer for peroxide may be used. As the stabilizer, one or more of phosphoric acid, sodium phosphate, sodium stannate, ammonium sulfate, sodium silicate, acetanilide, 8-hydroxyquinonline, pyridinecarboxylic acid, benzoic acids, nitrate salts, and the like may be used. The stabilizer may be included in the amount of 1 wt % or less, based on 100 wt % of the first contents. More preferably, the stabilizer may be included in the amount of 0.5 wt % or less, based on 100 wt % of the first contents.

The second contents may comprise a polishing agent to physically remove foreign substance or bacterial film on the surface of tooth. As the polishing agent, precipitated calcium carbonate, alumina, silicon dioxide, precipitated silica, and the like may be used, but it is not limited thereto, and commonly used polishing agents may be used. The polishing agent may be included in the amount of 5 to 20 wt %, based on 100 wt % of the second contents. More preferably, it may be included in the amount of 5 to 15 wt %. The content of the polishing agent may have an influence on the viscosity of a toothpaste composition and dischargeability from a container, and if it is greater than 20 wt %, it may be difficult to discharge from a dip tube container.

At least one of the first contents and second contents may further comprise a thickener to obtain viscosity suitable for use in a dip tube container. In case a thickener is further included in the first contents, the thickener may be selected considering compatibility with peroxide.

The thickener may be one or more of organic thickeners and inorganic thickeners. As the examples of the inorganic thickeners, silica, alumina, talc and calcium carbonate, and the like may be mentioned. As the silica, precipitated silica, fumed silica, and the like may be used. Preferably, the inorganic thickener may be fumed silica. As the examples of the organic thickeners, poloxamer such as poloxamer 407, xanthan gum, carbomer, carboxymethyl cellulose, polyacrylic acid, polyvinyl pyrrolidone, and polyethylene such as POLYOX™, hydroxypropyl methyl cellulose, hydroxypropyl cellulose, and the like may be mentioned.

In case inorganic thickener is used without organic thickener, the inorganic thickener may be included in the amount of 2 to 10 wt %, based on 100 wt % of the first or second contents. More preferably, it may be included in the amount of 2 to 8 wt %. If the content of the inorganic thickener is less than 2 wt %, the viscosity may be too low, and thus, shape retention suitable for use may not be secured, and if it is greater than 10 wt %, it may be difficult to discharge from a dip tube container. Rather than using an excessive amount of inorganic thickeners, it is preferable to use only organic thickeners, or use organic thickeners and inorganic thickeners in combination, and the organic thickeners may be included in the amount of 10 wt % or less, based on 100 wt % of the first or second contents. More preferably, the total weight of the organic thickeners and inorganic thickeners may be 20 wt % or less, based on 100 wt % of the first or second contents.

In the toothpaste composition according to the embodiment of the invention, the first contents and second contents may respectively comprise a thickener and a polishing agent in the above described contents, and thus, have viscosity of 5,000 to 40,000 cps, respectively. Preferably, the viscosity may be 10,000 to 30,000 cps. The viscosity of toothpaste directly connects with the storage method and container, and if the viscosity of the contents is less than 5,000 cps, the toothpaste may become liquid and may not have shape retention, and if it is greater than 40,000 cps, it may be suitable for a paste toothpaste, and it may be difficult to discharge from a dip tube container.

In case the viscosities of the first contents and second contents are different, the viscosity ratio may be also 1.2:1 to 1:1.2. Thus, when each contents are filled in a container having a diaphragm or separate containers, and then, a dip tube is operated to discharge them, they can be simultaneously discharged from a dip tube container and used because the dischargeabilities of two contents are similar.

At least one of the first contents and second contents further comprises a pharmaceutically effective agent for use in a toothpaste composition, additives for preventing coloration, a surfactant, ethanol, a catalyst, and the like.

The pharmaceutically effective agent may be sodium fluoride, sodium monofluorophosphate commonly used in a toothpaste composition.

The additives for preventing coloration may form a chelate with material inducing coloration, or remove attachment of corresponding material to teeth or dental calculus, and may be SAPP(sodium acid pyrophosphate), TSPP(tetrasodium pyrophosphate), TKPP(tetrapotassium phosphate), pentasodium triphosphate(TPP), SHMP(sodium hexametaphosphate), Sporix™(sodium metaphosphate), and the like.

Besides, the contents may further comprise surfactant such as sorbitan monooleate (span 80), and a pH controlling agent such as sodium citrate, citric acid monohydrate, phosphoric acid, and the like, and may further comprise saccharin or fragrance for improvement in sense of use. Ethanol may be included as a solvent for oil soluble ingredients such as fragrance, and may provide a feeling of refreshment when using toothpaste. In addition, the contents may further comprise tocopherol, charcoal, bamboo salt, Himalaya pink salt, and the like, and may provide antigingivitis effect.

The second contents which do not comprise peroxide may further comprise a catalyst for peroxide. The catalyst may be an activator for improving the activity of peroxide. As the first contents and second contents are mixed when used by the user, the catalyst in the second contents may improve whitening effect of peroxide in the first contents. The catalyst may be one or more selected from the group consisting of iron chloride(III)(FeCl₃), magnesium chloride(MgCl₂) and magnesium gluconate, and iron oxide(Fe₂O₃).

As explained, in the toothpaste composition according to the embodiment of the invention, the first contents comprise peroxide as a whitening agent, the second contents does not comprise peroxide but comprise a polishing agent, and at least one of the first contents and second contents further comprises a thickener, thus providing a toothpaste composition suitable for use in a dip tube container. Thus, before use by the user, the first contents and second contents are filled and stored in a container having a diaphragm or separate containers, and when brushing teeth, the dip tube container is simultaneously pumped to simultaneously discharge and use them.

Hereinafter, a toothpaste composition according to another embodiment of the invention will be explained. A difference in the main ingredients included in the first contents and second contents will be mainly explained, and the contents in common with the toothpaste composition according to the above described embodiment will be omitted.

A toothpaste composition according to another embodiment of the invention may comprise first contents comprising one or more ingredients selected from the group consisting of salts having divalent ions such as stannous fluoride, zinc chloride, strontium chloride, and calcium chloride, and the like, and second contents that form a complex with the above ingredients or inactivate the above ingredients.

Stannous fluoride is an ingredient having antigingivitis effect, and potassium nitrate, zinc chloride, calcium chloride and strontium chloride are ingredients having the effect of preventing or alleviating the symptoms of sensitive teeth. Although such ingredients respectively have useful effects for oral cavity health, they may induce coloration of teeth, and a chelating agent forming a complex with the above ingredients or water deactivating the above ingredients by hydrolysis, and the like, thus preventing coloration, may serve as a tooth whitening agent. Thus, in the toothpaste composition according to the embodiment of the invention, the first contents may be an oral composition comprising effective ingredients for oral cavity health, and the second contents may be an oral composition comprising a tooth whitening agent provided in case the above ingredients are used.

Specifically, the first contents may comprise stannous fluoride, and the second contents may comprise water in the amount of 20 to 60 wt %, based on 100 wt % of the second contents, and at least one of pyrophosphoric acid such as SAPP, TSPP, polyphosphoric acid such as SHMP, Sporix™, and pharmaceutically acceptable salts thereof. The second contents comprise water in the amount of 20 to 60 wt %, based on 100 wt % of the second contents, which corresponds to the level equivalent to common toothpaste. The first contents do not comprise water, or comprise water in the amount of 2 to 10 wt %, based on 100 wt % of the first contents, thus maintaining anhydrous or low water condition. In this case, the use may simultaneously discharge and use the first contents and second contents through a dip tube container, and stannous fluoride, after performing a function for antigingivitis effect, may form a complex with pyrophosphoric acid or may be hydrolyzed by water, and thus, may be removed without inducing coloration of teeth.

Specifically, the first contents may comprise one or more ingredients selected from the group consisting of zinc chloride, potassium nitrate, calcium chloride and strontium chloride, and the second contents may comprise one or more ingredients selected from the group consisting of disodium succinate, dicalcium oxalate, monopotassium phosphate, and dipotassium phosphate. In this case, the user may simultaneously discharge and use the first contents and second contents through a dip tube container.

Although zinc chloride, potassium nitrate, calcium chloride and strontium chloride in the first contents, and disodium succinate, dicalcium oxalate, monopotassium phosphate, and dipotassium phosphate in the second contents respectively have desensitization effect, in case the two contents are simultaneously discharged to form a complex, sensitive prevention and alleviation effect may be improved. For example, in case the first contents comprise zinc chloride, and the second contents comprises monopotassium phosphate, dipotassium phosphate, or a mixture thereof, an insoluble complex such silver phosphate may be formed to improve sensitive teeth prevention and alleviation effect.

In the toothpaste composition according to the embodiment of the invention, at least one of the first contents and second contents may comprise a thickener and a polishing agent as explained above, and thus, have viscosity and viscosity ratio as explained above.

As explained, in the toothpaste composition according to the embodiment of the invention, the first contents comprise ingredients having predetermined effects for oral cavity health, and the second contents comprise a chelating agent for the above ingredients as a whitening agent for preventing tooth coloration by the above ingredients, thus providing whitening effect and oral cavity health effect, and simultaneously, at least one of the first contents and second contents comprises at least one of a thickener and a polishing agent, thus providing a toothpaste composition suitable for use in a dip tube container. Thus, before user by the user, the first contents and second contents are filled and stored in a container having a diaphragm or separate containers, and when brushing teeth, the dip tube container may be simultaneously pumped to simultaneously discharge and use them.

Hereinafter, based on the contents of the above explained embodiments, more specific examples of the invention will be explained.

### Preparation of a Toothpaste Composition Comprising Peroxide

In order to evaluate storage stability, degree of hardening, and dischargeability from a dip tube container of a toothpaste composition comprising peroxide as a tooth whitening agent, contents A to J were prepared according to the composition (wt %) of the following Table 1.

TABLE 1

| | A | B | C | D | E | F | G | H | I | J |
|---|---|---|---|---|---|---|---|---|---|---|
| Glycerin | 50 | 45 | 45 | 20 | 20 | 20 | 40 | 50 | 50 | 10 |
| PEG300 | 1 | 0 | 0 | 0 | 0 | 10 | 5 | 1 | 1 | 1 |
| Precipitated | 0 | 14 | 15 | 25 | 0 | 0 | 20 | 2 | 14 | 14 |
| silica | | | | | | | | | | |
| Alumina | 0 | 0 | 20 | 0 | 0 | 0 | 0 | 0 | 0 | 0 |
| fumed silica | 6 | 0 | 0 | 0 | 0 | 4 | 0 | 10 | 0 | 0 |
| Sodium lauryl | 2 | 2 | 2 | 2 | 0 | 0 | 2 | 0 | 0 | 2 |
| sulfate | | | | | | | | | | |
| Sodium fluoride | 0 | 0.44 | 0.44 | 0.44 | 0 | 0 | 0.44 | 0 | 0 | 0.44 |
| Hydrogen | 6 | 0 | 0 | 0 | 6 | 0 | 0 | 6 | 6 | 3 |
| peroxide | | | | | | | | | | |
| Urea peroxide | 0 | 0 | 0 | 0 | 0 | 30 | 0 | 0 | 0 | 0 |
| SAPP | 0.2 | 0 | 0 | 0 | 0.05 | 0 | 0 | 0.2 | 0.2 | 0.2 |
| TSPP | 0 | 0.5 | 0.5 | 0.5 | 0 | 0 | 3 | 0 | 0 | 0.5 |
| SHMP | 0 | 0.2 | 0.2 | 0.2 | 0 | 0 | 3 | 0 | 0 | 0 |
| Sporix | 0.25 | 0 | 0 | 0 | 0 | 0 | 0 | 0.25 | 0.25 | 0.25 |
| PVP | 5 | 0 | 0 | 0 | 0 | 10 | 0 | 5 | 5 | 0 |
| poloxamer407 | 0.5 | 0 | 0 | 0 | 0 | 0 | 0 | 0.5 | 0.5 | 0 |
| Xanthan gum | 1.4 | 0.8 | 0.8 | 0.2 | 0 | 0 | 0.8 | 1.4 | 1.4 | 0.8 |
| CMC | 0 | 0.6 | 0.6 | 1.3 | 0 | 0 | 0 | 0 | 0 | 0.6 |
| Carbomer | 0 | 0 | 0 | 0 | 1.8 | 0 | 0 | 0 | 0 | 0 |
| PAA | 0 | 0 | 0 | 0 | 0 | 0 | 0.05 | 0 | 0 | 0 |
| HPMC | 0.1 | 0 | 0 | 0 | 0 | 0.5 | 0 | 0.1 | 0.1 | 0 |
| HPC | 0.1 | 0 | 0 | 0 | 0 | 0 | 0 | 0.1 | 0.1 | 0 |
| Sodium citrate | 0.2 | 0 | 0 | 0 | 0 | 0.2 | 0 | 0.2 | 0.2 | 0 |
| Citric acid | 0.07 | 0 | 0 | 0 | 0 | 0 | 0 | 0.07 | 0.07 | 0 |
| monohydrate | | | | | | | | | | |
| phosphoric | 0.2 | 0 | 0 | 0 | 0.05 | 0.05 | 0 | 0.2 | 0.2 | 0 |
| acid | | | | | | | | | | |
| Span 80 | 0.2 | 0 | 0 | 0 | 0 | 0 | 0 | 0.2 | 0.2 | 0.2 |
| FeCl3 | 0 | 0.1 | 0.1 | 0.1 | 0 | 0 | 0.1 | 0 | 0 | 0.1 |
| Sucralose | 0 | 0.05 | 0 | 0 | 0 | 0 | 0 | 0 | 0 | 0.05 |
| sodium | 0.3 | 0.24 | 0.24 | 0.24 | 0.05 | 0.05 | 0.24 | 0.3 | 0.3 | 0.3 |
| saccharin | | | | | | | | | | |
| Fragrance | 1.2 | 1.2 | 1.2 | 1.2 | 0.5 | 0.5 | 1.2 | 1.2 | 1.2 | 1.2 |
| Ethanol | 0 | 0 | 0 | 0 | 10 | 20 | 0 | 0 | 0 | 0 |
| Tribasic sodium | 0 | 0 | 0 | 0 | 0.25 | 0.01 | 1 | 0 | 0 | 0 |
| phosphate | | | | | | | | | | |
| Purified water | Remaining | Remaining | Remaining | Remaining | Remaining | Remaining | Remaining | Remaining | Remaining | Remaining |
| | amount | amount | amount | amount | amount | amount | amount | amount | amount | amount |

A dip tube type two-chamber container was prepared, and the contents A to J were respectively put in chamber 1 and chamber 2 according to the combination of the following Table 2, thus preparing Examples 1 to 5 and Comparative Examples 1 to 5. Since the physical properties of the toothpaste composition and the properties of the ingredients in the toothpaste composition at the time of final use vary according to the combination of the contents stored in two chambers, various Examples and Comparative Examples were prepared as shown in the following Table 2, so as to evaluate physical properties and characteristics according to various combinations.

In each Example and Comparative Example, the contents put in the chamber 1 were prepared according to a common gel preparation method, and the contents put in the chamber 2 were prepared by dispersing and dissolving polyhydric alcohol, additives, surfactant, saccharin, and the like in water or mixing them, and then, adding a polishing agent such as silica and mixing under vacuum.

TABLE 2

| | Example 1 | Example 2 | Example 3 | Example 4 | Example 5 |
|---|---|---|---|---|---|
| Chamber 1 | A | E | F | A | E |
| Chamber 2 | B | B | B | G | G |
| | Comparative | Comparative | Comparative | Comparative | Comparative |
| | Example 1 | Example 2 | Example 3 | Example 4 | Example 5 |
| Chamber 1 | I | A | A | H | J |
| Chamber 2 | B | C | D | B | J |

### Experimental Example 1. Evaluation of the Physical Properties of Toothpaste Compositions Comprising Peroxide

### Experimental Example 1-1. Confirmation of Storage Stability, Degree of Hardening, and Dischargeability of Toothpaste Compositions

While storing the toothpaste compositions according to Examples 1 to 4 and Comparative Examples 1, 4 and 5 respectively contained in a dip tube container at 50° C. for 4 weeks, the remaining peroxide rate was derived by titration of peroxide included in the toothpaste composition, thus confirming storage stability of the toothpaste compositions.

Simultaneously, it was observed with an unaided eye whether or not the toothpaste compositions hardened, and after 4 weeks, the dip tube container was pumped to confirm dischargeability, and the results were shown in the following Table 3.

TABLE 3

| | | | | | Comparative | Comparative | Comparative |
|---|---|---|---|---|---|---|---|
| | Example 1 | Example 2 | Example 3 | Example 4 | Example 1 | Example 4 | Example 5 |
| 2 weeks remaining | 95% | 96% | 94% | 94% | 75% | 95% | 70% |
| peroxide rate | | | | | | | |
| 4 weeks remaining | 91% | 92% | 92% | 90% | 48% | 91% | 40% |
| peroxide rate | | | | | | | |
| Degree of hardening | Not | Not | Not | Not | Not | Not | Harden |
| of toothpaste after | harden | harden | harden | harden | harden | harden | |
| storage for 4 weeks | | | | | | | |
| Dischargeability of | Easy to | Easy to | Easy to | Easy to | Easy to | Cannot be | Cannot be |
| toothpaste after | simultaneously | simultaneously | simultaneously | simultaneously | simultaneously | simultaneously | simultaneously |
| storage for 4 weeks | discharge | discharge | discharge | discharge | discharge | discharged | discharged |

In the case of Examples 1 to 4, remaining peroxide rate was high even if time elapsed, and it was confirmed that storage was excellent and simultaneous discharge was easy.

In the case of Comparative Example 1 wherein peroxide and a polishing agent are included in the contents of the same chamber, and in the case of Comparative Example 5 wherein peroxide and a peroxide activator are included in the contents of the same chamber, remaining peroxide rate rapidly decreased as time elapsed, indicating that whitening effect may be lowered.

In Comparative Example 4, fumed silica having excellent compatibility with peroxide was used as inorganic thickener and remaining peroxide rate was high, but since the inorganic thickener fumed silica was used 12% excessive, it was impossible to simultaneously discharge.

The degree of hardening and storage property of toothpaste are also related to the content of polyol in the contents, and in Comparative Example 5 having low polyol content, storage property was inferior, and thus, the toothpaste hardened during storage, and it was difficult to simultaneously discharge.

### Experimental Example 1-2. Confirmation of Storage Stability, Degree of Hardening and Dischargeability of Toothpaste Compositions

While storing the toothpaste compositions according to Examples 1 and 5 and Comparative Examples 2 and 3 at 25° C. for 4 hours, the viscosity of each contents was confirmed. After filling a 100 mL plastic container with the contents, it was stored in an incubator of 25° C., and rotated 5 turns at 20 rpm using spindle(Brookfield RVT type No. 5 or No. 7) to measure the viscosity value(cps), and the results were shown in the following Table 4.

TABLE 4

| | | | | | Comparative | | Comparative | |
|---|---|---|---|---|---|---|---|---|
| | Example 1 | | Example 5 | | Example 2 | | Example 3 | |
| | A | B | E | G | A | C | A | D |
| 25° C. | 10,000 | 10,000 | 35,000 | 35,000 | 10,000 | 100,000 | 10,000 | 70,000 |
| Initial viscosity | | | | | | | | |
| 25° C. | 20,000 | 20,000 | 38,000 | 38,000 | 20,000 | 120,000 | 20,000 | 90,000 |
| 4 week viscosity | | | | | | | | |

In the case of Comparative Examples 2 and 3 having the initial viscosities of 70,000 to 100,000 cps corresponding to the viscosity of common paste toothpaste, it was confirmed that the degree of viscosity increase over time was higher, compared to Examples 1 and 5 having the initial viscosities of 10,000 to 35,000 cps. In Examples 1 and 5, the viscosity increased just 3,000 to 10,000 cps, while in Comparative Examples 2 and 3, the viscosity increased 20,000 cps, and thus, the viscosity increase was more than 2 times in Comparative Examples. The above results show that during storage, as time elapsed, the degree of viscosity increase was relatively large in the contents (Comparative Examples 2 and 3) using excessive amount of polishing agents.

### Experimental Examples 1-3. Evaluation of Sense of Use of Toothpaste Compositions

The sense of use was evaluated by 10 consumers who brushed teeth three time a day for one week, using the toothpaste compositions according to Examples 1 and 5 and Comparative Examples 2 and 3. Pumpability, simultaneous dischargeability, whitening effect felt, irritation and the whole product satisfaction were surveyed.

The pumpability was evaluated by satisfaction felt when pushing the pump with hand to squeeze toothpaste on toothbrush, and loosening was evaluated by how fast and easy the toothpaste is loosened without mass in the oral cavity after squeezing toothpaste on toothbrush and brushing teeth. The items except irritation was evaluated with a perfect score of 5 points (5 points: very excellent, 4 points: excellent, 3 points: average, 2 points: unsatisfactory, 1 point: bad), and the mean value was calculated. The irritation was evaluated as 5 points being the level of toothpaste without peroxide, 4 points being less irritable than peroxide toothpaste, 3 points being the level of 3% peroxide toothpaste (Crest 3D White Brilliance 2-step peroxide gel), 2 points being slightly irritable level, and 1 point being severe irritation level, and the mean value was calculated.

TABLE 5

| | | | Comparative | Comparative |
|---|---|---|---|---|
| | Example 1 | Example 5 | Example 2 | Example 3 |
| Pumpability | 3.6 | 3.5 | 3 | 2.8 |
| Simultaneous | 5 | 4.5 | 2 | 2.5 |
| dischargeability | | | | |
| Simultaneous loosening | 4.0 | 4.0 | 2.0 | 2.5 |
| Whitening effect | 3.8 | 3.5 | 3 | 3.5 |
| Irritation before/after | 3.5 | 4.0 | 2.0 | 2.5 |
| use | | | | |
| Product satisfaction | 3.8 | 4.0 | 2.0 | 2.5 |

The toothpaste compositions according to Examples 1 and 5 have excellent pumpability and simultaneous dischargeability. And, loosening, the degree of two contents being sufficiently loosened in mouth, was also excellent, and although comprising 3 wt % peroxide, irritation was lower than commercial 3% peroxide toothpaste.

In the toothpaste compositions according to Comparative Examples 2 and 3, the viscosity of the contents in the chamber 1 comprising peroxide was much lower than the viscosity of the chamber 2, and thus, it was confirmed that simultaneous dischargeability was bad, and since the contents of the chamber 1 was discharged more, irritation was higher.

### Preparation of Whitening Toothpaste Composition Comprising Antigingivitis Ingredients

Toothpaste compositions comprising stannous fluoride as antigingivitis ingredients were prepared with the composition (wt %) of the following Table 6. As Comparative Examples, low water toothpaste having purified water content less than 10 wt % and common toothpaste having purified water content of 20 wt % or more were prepared.

TABLE 6

| | | | | | | | Comparative | Comparative |
|---|---|---|---|---|---|---|---|---|
| | | | | | | | Example 6 | Example 7 |
| | Example 6 | | Example 7 | | Example 8 | | Low water | Common |
| | Chamber 1 | Chamber 2 | Chamber 1 | Chamber 2 | Chamber 1 | Chamber 2 | toothpaste | toothpaste |
| Glycerin | 50 | 20 | 50 | 20 | 46 | 20 | 38 | 20 |
| PEG300 | 23 | 5 | 20 | 5 | 25 | 5 | 20 | 5 |
| Precipitated | 16 | 16 | 15 | 15 | 14 | 14 | 25 | 20 |
| silica | | | | | | | | |
| Sodium lauryl | 2 | 2 | 2 | 0 | 2 | 0 | 2 | 2 |
| sulfate | | | | | | | | |
| Stannous Fluoride | 0.80 | 0 | 0.80 | 0 | 0.80 | 0 | 0.40 | 0.40 |
| SAPP | 0 | 6 | 0 | 0 | 0 | 4 | 0 | 0 |
| TSPP | 0 | 0 | 0 | 4 | 0 | 0 | 0 | 0 |
| SHMP | 0 | 0 | 0 | 0 | 0 | 2 | 6 | 6 |
| poloxamer407 | 0.05 | 0.05 | 0.05 | 0.05 | 0.05 | 0.05 | 0.05 | 0.05 |
| Xanthan gum | 0.2 | 0.8 | 0.2 | 0.8 | 0.2 | 0.8 | 0.2 | 0.2 |
| CMC | 0 | 0.1 | 0 | 0.1 | 0 | 0.1 | 0 | 1.2 |
| Carrageenan | 0.5 | 0.2 | 0.4 | 0.2 | 0.5 | 0.2 | 0.4 | 0 |
| HPMC | 0 | 0 | 0 | 0 | 0 | 0.5 | 0 | 0 |
| HPC | 0 | 0 | 0 | 0 | 0 | 0 | 0 | 0 |
| Sodium citrate | 0.2 | 0.2 | 0.2 | 0.2 | 0.2 | 0.2 | 0.2 | 0.2 |
| Citric acid | 0.07 | 0.07 | 0.07 | 0.07 | 0.07 | 0.07 | 0.07 | 0.07 |
| monohydrate | | | | | | | | |
| phosphoric acid | 0 | 0 | 0 | 0.05 | 0 | 0.05 | 0 | 0 |
| Span 80 | 0.2 | 0 | 0 | 0 | 0 | 0 | 0.2 | 0 |
| Sucralose | 0.04 | 0.04 | 0.04 | 0.04 | 0.04 | 0.04 | 0.04 | 0.04 |
| sodium saccharin | 0.24 | 0.24 | 0.24 | 0.24 | 0.24 | 0.24 | 0.24 | 0.24 |
| Fragrance | 1.2 | 1.2 | 1.2 | 1.2 | 1.2 | 1.2 | 1.2 | 1.2 |
| Tribasic sodium | 0.3 | 0.25 | 0.3 | 0.25 | 0.3 | 0.25 | 0.3 | 0.25 |
| phosphate | | | | | | | | |
| Purified water | Remaining | Remaining | Remaining | Remaining | Remaining | Remaining | Remaining | Remaining |
| | amount | amount | amount | amount | amount | amount | amount | amount |

### Experimental Example 2. Evaluation of Sense of Use of Toothpaste Compositions

The toothpaste compositions according to Examples 6 to 8 were respectively put in a two-chamber container, and for the toothpaste compositions according to Comparative Examples 6 and 7, the same contents were respectively put in a two-chamber container, and then, the sense of use was evaluated by 10 consumers with gingival bleeding when brushing teeth, who brushed teeth three times a day for one month.

The sense of use of toothpaste, gingival bleeding decrease effect, whitening effect felt, and the whole product satisfaction were surveyed. These were evaluated with a perfect score of 5 points (5 points: very excellent, 4 points: excellent, 3 points: average, 2 points: unsatisfactory, 1 point: bad), and the mean values were calculated.

TABLE 7

| | | | | Comparative | Comparative |
|---|---|---|---|---|---|
| | Example 6 | Example 7 | Example 8 | Example 6 | Example 7 |
| Sense of use | 3.8 | 4.0 | 3.8 | 2.5 | 3.8 |
| Gingival bleeding | 3.5 | 3.5 | 3.5 | 3.5 | 3.0 |
| decrease effect | | | | | |
| Whitening effect felt | 3.5 | 3.5 | 3.5 | 3.5 | 2.5 |
| Product satisfaction | 3.5 | 3.5 | 3.5 | 2.9 | 2.8 |

The users responded that when using the toothpaste compositions according to Examples 6 to 8, gingival bleeding decreased and whitening effect was felt.

In the case of low water toothpaste according to Comparative Example 6, although it has gingival bleeding decrease effect due to stannous fluoride ingredient, dischargeability was bad, and thus, sense of use and product satisfaction were low.

In the case of common toothpaste according to Comparative Example 7, although it has average level of gingival bleeding decrease effect, it was difficult to feel whitening effect.

### Preparation of Whitening Toothpaste Compositions Comprising Desensitizer Ingredients

Toothpaste compositions consisting of contents comprising zinc chloride as a desensitizer ingredient and contents comprising a chelating agent thereof were prepared with the compositions (wt %) according to the following Table 8. As Comparative Examples, toothpaste compositions comprising zinc chloride, sodium phosphate, potassium phosphate, sodium succinate, and the like in one contents were prepared and put in a tube.

TABLE 8

| | Example 9 | | Example 10 | | Comparative | Comparative |
|---|---|---|---|---|---|---|
| | Chamber 1 | Chamber 2 | Chamber 1 | Chamber 2 | Example 8 | Example 9 |
| Glycerin | 40 | 40 | 40 | 40 | 40 | 40 |
| PEG300 | 5 | 5 | 5 | 5 | 5 | 5 |
| Precipitated silica | 15 | 15 | 15 | 15 | 15 | 15 |
| Sodium lauryl | 2 | 2 | 2 | 2 | 2 | 2 |
| sulfate | | | | | | |
| Zinc chloride | 0.18 | 0 | 0.18 | 0 | 0.09 | 0.09 |
| Sodium | 0 | 1.52 | 0 | 1.52 | 0.76 | 0.76 |
| monofluorophosphate | | | | | | |
| Monobasic potassium | 0 | 0.05 | 0 | 0.05 | 0.05 | 0.05 |
| phosphate | | | | | | |
| Dibasic potassium | 0 | 1.5 | 0 | 1.5 | 0.75 | 0.75 |
| phosphate | | | | | | |
| Disodium succinate | 0 | 2.4 | 0 | 0 | 1.2 | 0 |
| Dipotassium oxalate | 0 | 0 | 0 | 2.4 | 0 | 1.2 |
| Xanthan gum | 0.5 | 0.5 | 0.5 | 0.5 | 0.5 | 0.5 |
| CMC | 0.8 | 0.8 | 0.8 | 0.8 | 0.8 | 0.8 |
| Sodium citrate | 0.2 | 0.2 | 0.2 | 0.2 | 0.2 | 0.2 |
| Citric acid | 0.07 | 0.07 | 0.07 | 0.07 | 0.07 | 0.07 |
| monohydrate | | | | | | |
| Span 80 | 0.2 | 0.2 | 0.2 | 0.2 | 0.2 | 0.2 |
| sucralose | 0.05 | 0.05 | 0.05 | 0.05 | 0.05 | 0.05 |
| sodium saccharin | 0.24 | 0.24 | 0.24 | 0.24 | 0.24 | 0.24 |
| fragrance | 1.2 | 1.2 | 1.2 | 1.2 | 1.2 | 1.2 |
| Tribasic sodium | 0.2 | 0.2 | 0.2 | 0.2 | 0.2 | 0.2 |
| phosphate | | | | | | |
| Purified water | Remaining | Remaining | Remaining | Remaining | Remaining | Remaining |
| | amount | amount | amount | amount | amount | amount |

### Experimental Example 3. Confirmation of Storage Stability of Toothpaste Compositions

The toothpaste compositions according to Examples 9 and 10 were put in dip tube type two-chamber containers, the toothpaste compositions according to Comparative Examples 8 and 9 were put in the existing tubes, and then, while storing at 50° C. for 4 weeks, it was observed whether or not precipitate was formed inside of each container.

TABLE 9

| | | | Comparative | Comparative |
|---|---|---|---|---|
| | Example 9 | Example 10 | Example 8 | Example 9 |
| 50° C. | No precipitate | No precipitate | No precipitate | Precipitation |
| 1 week elapsed | | | | |
| 50° C. | No precipitate | No precipitate | Precipitated | Precipitated |
| 2 weeks elapsed | | | | |
| 50° C. | No precipitate | No precipitate | Precipitated | Precipitated |
| 4 weeks elapsed | | | | |

The above experimental results show the need and effect of separate storage such as in two chambers when storing zinc chloride and a chelating agent for zinc chloride, for a toothpaste composition comprising zinc chloride as a desensitizer ingredient.

The present invention is not limited to the above explained examples, and the present invention may include combinations of the examples or combinations of at least one of the examples and known technology as another example.

Although the present invention has been explained in detail through specific examples, these are presented to specifically explain the present invention, the present invention is not limited thereby, and it is obvious that modifications or improvement can be made by one of ordinary knowledge in the art within the technical ideas of the present invention.

Simple modifications or alterations of the invention are within the scope of the present invention, and specific range of protection of the invention is determined by the attached claims.

[Reference Numerals]

| | |
|---|---|
| 1: two-chamber container | 10: container body |
| 11: diaphragm | 12: first container |
| 13: second container | 14: protrusion part |
| 15: coupling home | 20: fixing member |
| 21: cap | 22: spring |
| 30: nozzle | 31: outlet |
| 40: pump | 50: tube |
| 2: two-chamber container | 60: first container |
| 61: first discharge part | 62: first outlet |
| 70: cap | 80: second container |
| 81: second discharge part | 82: second outlet |

1. A toothpaste composition for tooth whitening comprising:
first contents; and
second contents,
wherein one of the first contents and second contents comprises a tooth whitening agent, the first contents and second contents being filled in a container having a diaphragm in a single container body or a container having separate container bodies and simultaneously used when brushing teeth, and
wherein the container is a dip tube container.

2. The toothpaste composition according to claim 1,
wherein the first contents comprise peroxide, and the second contents comprise a polishing agent.

3. The toothpaste composition according to claim 2,
wherein the peroxide is one or more selected from the group cons g of urea peroxide, sodium peroxide, calcium peroxide and hydrogen peroxide.

4. The toothpaste composition according to claim 1,
wherein at least one of the first contents and die second contents comprises a thickener.

5. The toothpaste composition according to claim 4,
wherein at least one of the first contents and the second contents comprises the thickener ire an amount of 20 wt % or less, based on 100 wt % of each contents.

6. The toothpaste composition according to claim 4,
wherein the thickener is one or more selected from the group consisting of precipitated silica, fumed silica, alumina, talc, calcium carbonate, poloxamer, xanthan gum, carbomer, carboxymethyl cellulose, polyacrylic acid, polyvinyl pyrrolidone, polyethylene, hydroxypropyl methylcellulose, and hydroxypropyl cellulose.

7. The toothpaste composition according to claim 2,
wherein the second contents comprise the polishing agent in an amount 5 to 20 wt %, based on 100 wt % of the second contents.

8. The toothpaste composition according to claim 1,
wherein the first contents comprise one or more selected from the group consisting of stannous fluoride, zinc chloride, potassium nitrate, calcium chloride and strontium chloride, and the second contents form a complex with the said first contents or inactivate said first contents.

9. The toothpaste composition according to claim 8,
wherein the first contents comprise stannous fluoride, and
the second contents comprise (a) at least one selected from the group consisting of pyrophosphoric acid, polyphosphoric acid, and pharmaceutically acceptable salts thereof and (b) water, wherein the water is present in the second contents in an amount of 20 to 60 wt %, based on 100 wt % of the second contents.

10. The toothpaste composition according to claim 9,
wherein the first contents further comprise water, wherein the water is present in the first contents in an amount of 2 to 10 wt %, based on 100 wt % of the first contents.

11. The toothpaste composition according to claim 9,
wherein the second contents comprise at least one selected from the group consisting of pyrophosphoric acid, polyphosphoric acid, and pharmaceutically acceptable salts thereof in an amount of 2 to 12 wt %, based on 100 wt % of the second contents.

12. The toothpaste composition according to claim 8,
wherein the first contents comprise one or more selected from the group consisting of zinc chloride, potassium nitrate, calcium chloride and strontium chloride, and
the second contents comprise one or more selected from the group consisting of disodium succinate, dicalcium oxalate, monopotassium phosphate, and dipotassium phosphate.

13. The toothpaste composition according to claim 1,
wherein viscosities of the first contents and the second contents are 5,000 to 40,000 cps.

14. The toothpaste composition according to claim 13,
wherein a ratio of the viscosities of the first contents and the second contents is 12:1 to 1:1.2.

15. A dispensing container providing two storage spaces for a toothpaste composition for tooth whitening having first and second contents, the dispensing container comprising:
a tube in communication with each of the two storage spaces;
a pump connected to each tube; and
a nozzle for discharging the first and second contents of the toothpaste composition,
wherein the dispensing container a dip tube container.

16. The dispensing container according to claim 15, further comprising a diaphragm for dividing an internal space of the dispensing container into the two storages spaces, the two storage spaces being a first storage space and a second storage space,
wherein the first contents and the second contents are respectively stored in the first storage space and the second storage space divided by the diaphragm.

17. The dispensing container according to claim 15,
wherein the dispensing container comprises a first container body in which the first contents are stored, and a second container body in which the second contents are stored.

18. The dispensing container according to claim 17,
wherein the first and second container bodies are disposed in an internal space of the dispensing container.

19. The dispensing container according to claim 17,
wherein second container body is disposed in the first container body.

20. (canceled)

21. A dispensing container for a toothpaste composition for tooth whitening having first and second contents, the dispensing container comprising:
a first container body having a first storage space for the first contents, the first container body having a first discharging part to discharge the first contents; and
a second container body having a second storage space for the second storage contents, the second container body having a second discharging part to discharge the second contents, the second container body being located in the first container body such that the second contents are also discharged through the first discharging part.
